(19) United States
(12) Patent Application Publication
(10) Pub. No.: US 2026/0335399 A1
(43) Pub. Date: Oct. 29, 2026

(54) BAG3 METHODS AND USES FOR TREATMENT OF CARDIAC AMYLOIDOSIS

(71) Applicant: TEMPLE UNIVERSITY OF THE COMMONWEALTH SYSTEM OF HIGHER EDUCATION, Philadelphia, PA (US)

(72) Inventor: Arthur M. FELDMAN, Wynnewood, PA (US)

(73) Assignee: TEMPLE UNIVERSITY OF THE COMMONWEALTH SYSTEM OF HIGHER EDUCATION, Philadelphia, PA (US)

(21) Appl. No.: 19/111,569

(22) PCT Filed: Sep. 14, 2023
(86) PCT No.: PCT/US2023/074258

§ 371 Date: Mar. 13, 2025

Related U.S. Application Data
(60) Provisional application No. 63/376,014, filed on Sep. 16, 2022.

(51) Int. Cl. A61K 38/17; A61K 48/00; C12N 15/86

(52) U.S. Cl. CPC A61K 38/1709; A61K 48/0058; C12N 15/86; C12N 2750/14143; C12N 2830/50

(57) ABSTRACT

Heart failure WITH Preserved Ejection Fraction (HFpEF) effects approximately three million people in the United States.

### RELATED APPLICATIONS

This application claims priority to U.S. Provisional Patent Application No. 63/376,014, filed Sep. 16, 2022. The entire content of the foregoing application is incorporated herein by reference, including all text, tables, sequence listings and drawings.

### INTRODUCTION

Heart failure WITH Preserved Ejection Fraction (HFpEF) effects approximately three million people in the United States. Of these affected individuals, approximately 15 percent have increased cardiac amyloid. Furthermore, approximately half of cases of cardiac amyloidosis is of non-familial origin or cause.

### SUMMARY

It has now been discovered that decreased BCL2-Associated Athanogene 3 (BAG3) levels or activity give rise to a marked increase in beta amyloid levels cells and tissues, such as in heart, liver, skeletal muscle and central nervous system (CNS) cells or tissues. Accordingly, in certain embodiments, a method of increasing BAG3 levels or activity in a subject in need thereof comprises administering to the patient a therapeutically effective amount of an agent wherein the agent modulates expression or amount of BAG3 molecules, proteins or peptides thereof in a target cell or tissue. In certain embodiments, a method of treating a subject having cardiac disease, such as HFpEF, wherein said subject has at least one Bcl2-associated anthanogene 3 (BAG3) nucleotide variant (NV) in-frame insertion as compared to a control BAG3 nucleic acid sequence, comprises administering to the subject a therapeutically effective amount of an agent wherein the agent modulates expression or amount of BAG3 molecules, proteins or peptides thereof in a target cell or tissue. In certain embodiments, a method of treating a subject having a neurodegenerative disease associated with increased APP or β-amyloid levels, wherein said subject has at least one Bcl2-associated anthanogene 3 (BAG3) nucleotide variant (NV) in-frame insertion as compared to a control BAG3 nucleic acid sequence, comprises administering to the subject a therapeutically effective amount of an agent wherein the agent modulates expression or amount of BAG3 molecules, proteins or peptides thereof in a target cell or tissue, thereby decreasing APP or β-amyloid levels in the subject. In certain embodiments, a method of treating a subject having a neurodegenerative disease associated with increased APP or β-amyloid levels, wherein said subject has at least one Bcl2-associated anthanogene 3 (BAG3) nucleotide variant (NV) in-frame insertion as compared to a control BAG3 nucleic acid sequence, comprises administering to the subject a therapeutically effective amount of an agent wherein the agent modulates expression or amount of BAG3 molecules, proteins or peptides thereof in a target cell or tissue, thereby decreasing APP or β-amyloid levels and treating the disease in the subject.

In certain embodiments, a method of treating a patient suffering from, or, at risk of developing amyloidosis comprises administering to the patient a therapeutically effective amount of an agent wherein the agent modulates expression or amount of BCL2-associated athanogene 3 (BAG3) encoding nucleic acid, BAG3 protein or BAG3 peptide thereby treating amyloidosis. In certain embodiments, the the BAG3 encoding nucleic acid comprises an expression vector expressing a BAG3 protein or active fragment thereof.

In certain embodiments, a method of treating a patient suffering from, or, at risk of developing amyloidosis comprises administering to the patient a therapeutically effective amount of an agent wherein the agent modulates expression or amount of BCL2-associated athanogene 3 (BAG3) encoding nucleic acid, BAG3 protein or BAG3 peptide thereby treating amyloidosis, wherein the BAG3 encoding nucleic acid comprises an expression vector expressing a BAG3 protein or active fragment thereof, wherein the expression vector further comprises a promoter, the promoter comprising an inducible promoter, a constitutive promoter, bicistronic promoter or tissue specific promoter. In certain embodiments, the expression vector comprises a viral vector, cardiotropic vector, plasmid, or a yeast vector. In certain embodiments the expression vector is a pseudotyped viral vector.

In certain embodiments, a method of treating a patient suffering from, or, at risk of developing amyloidosis comprises administering to the patient a therapeutically effective amount of an agent wherein the agent modulates expression or amount of BCL2-associated athanogene 3 (BAG3) encoding nucleic acid, BAG3 protein or BAG3 peptide thereby treating amyloidosis, wherein a cardiotropic vector comprises an adenovirus vector, an adeno-associated virus vector (AAV), a coxsackie virus vector, cytomegalovirus vector, Epstein-Barr virus vector, parvovirus vector, or hepatitis virus vectors.

In certain embodiments, the AAV vector comprises a capsid protein having 90% or more sequence identity to any of AAV1, AAV2, AAV3, AAV4, AAV5, AAV6, AAV7m AAV8, AAV9, AAV10, AAV11 or AAV12.

In certain embodiments, a method of treating a patient suffering from, or, at risk of developing amyloidosis comprises administering to the patient a therapeutically effective amount of an agent wherein the agent modulates expression or amount of BCL2-associated athanogene 3 (BAG3) encoding nucleic acid, BAG3 protein or BAG3 peptide thereby treating amyloidosis, wherein the patient has or is at risk of having heart failure with preserved ejection fraction (HFpEF). In certain embodiments, the patient has or is at risk of having cardiac amyloid deposits or increased cardiac amyloid compared to age, gender and/or race matched control.

In certain embodiments, a method of treating a patient suffering from, or, at risk of developing amyloidosis comprises administering to the patient a therapeutically effective amount of an agent wherein the agent modulates expression or amount of BCL2-associated athanogene 3 (BAG3) encoding nucleic acid, BAG3 protein or BAG3 peptide thereby treating amyloidosis, wherein the patient expresses lower than normal levels of BAB3 in cardiac tissue.

In certain embodiments, a method of treating a patient suffering from, or, at risk of developing amyloidosis comprises administering to the patient a therapeutically effective amount of an agent wherein the agent modulates expression or amount of BCL2-associated athanogene 3 (BAG3) encoding nucleic acid, BAG3 protein or BAG3 peptide thereby treating amyloidosis wherein the amyloidosis comprises cardiac amyloidosis or Alzheimer's disease. In certain embodiments, wherein the patient does not have a mutation or defect in the transthyreitn (ATTAR) gene.

In certain embodiments, a method of treating a patient suffering from, or, at risk of developing amyloidosis comprises administering to the patient a therapeutically effective amount of an agent wherein the agent modulates expression or amount of BCL2-associated athanogene 3 (BAG3) encoding nucleic acid, BAG3 protein or BAG3 peptide thereby treating amyloidosis, wherein the BAG3 encoding nucleic acid comprises an expression vector expressing a BAG3 protein or active fragment thereof, wherein the expression vector further comprises a promoter, the promoter optionally comprising an inducible promoter, a constitutive promoter, bicistronic promoter or tissue specific promoter. In certain embodiments, the inducible promoter confers expression in cardiac tissue or CNS.

In certain embodiments, a method of treating a patient suffering from, or, at risk of developing amyloidosis comprises administering to the patient a therapeutically effective amount of an agent wherein the agent modulates expression or amount of BCL2-associated athanogene 3(BAG3) encoding nucleic acid, BAG3 protein or BAG3 peptide thereby treating amyloidosis, wherein the BAG3 encoding nucleic acid comprises an expression vector expressing a BAG3 protein or active fragment thereof, wherein the expression vector further comprises an AAV inverted terminal repeat (ITR).

In certain embodiments, a method of treating a patient suffering from, or, at risk of developing amyloidosis comprises administering to the patient a therapeutically effective amount of an agent wherein the agent modulates expression or amount of BCL2-associated athanogene 3 (BAG3) encoding nucleic acid, BAG3 protein or BAG3 peptide thereby treating amyloidosis, wherein the BAG3 encoding nucleic acid comprises an expression vector expressing a BAG3 protein or active fragment thereof, wherein the expression vector further comprises a polyadenylation sequence and/or stop codon.

### BRIEF DESCRIPTION OF THE DRAWINGS

FIGS. 1A-1B show confocal images of cardiac cells from BAG3 haploinsufficient (BAG3+/−) mice that experienced heart failure with preserved ejection fraction (HFpEF). The cells have been stained to visualize either APP or β-amyoid, as indicated.

FIGS. 2A-2B provide quantification of the amount of β-amyloid in heart cells of either BAG3+/− mice or wild-type mice, both of which experienced (HFpEF).

### DETAILED DESCRIPTION

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, certain materials and methods are described herein. In describing and claiming the present invention, the following terminology will be used.

The articles “a” and “an” are used herein to refer to one or to more than one (i.e., to at least one) of the grammatical object of the article. By way of example, “an element” means one element or more than one element. Thus, recitation of “a cell”, for example, includes a plurality of the cells of the same type. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

“About” as used herein when referring to a measurable value such as an amount, a temporal duration, and the like, is meant to encompass variations of +/−20%, +/−10%, +/−5%, +/−1%, or +/−0.1% from the specified value, as such variations are appropriate to perform the disclosed methods. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

As used herein “BAG3”, “BAG3 molecules”, “BCL2-associated athanogene 3 (BAG3) genes”, “BCL2-associated athanogene 3 (BAG3) molecules” are inclusive of all family members, mutants, cDNA sequences, alleles, fragments, species, coding and noncoding sequences, sense and antisense polynucleotide strands, etc. (HGNC (939) Entrez Gene (9531) Ensembl (ENSG00000151929) OMIM (603883) UniProtKB (095817)). Similarly, “BAG3”, “BAG3 molecules”, “BCL2-associated athanogene 3 (BAG3) molecules” also refer to BAG3 polypeptides or fragment thereof, proteins, variants, derivatives etc. The term “molecule”, thus encompasses both the nucleic acid sequences and amino acid sequences of BAG3.

Bcl-2 associated anthanogene-3 (BAG3), also known as BCL2-Associated Athanogene 3; MFM6; Bcl-2-Binding Protein Bis; CAIR-1; Docking Protein CAIR-1; BAG Family Molecular Chaperone Regulator 3; BAG-3; BCL2-Binding Athanogene 3; or BIS, is a cytoprotective polypeptide that competes with Hip-1 for binding to HSP 70. The NCBI reference amino acid sequence for BAG3 can be found at Genbank under accession number NP_004272.2; Public GI: 14043024. The amino acid sequence of Genbank accession number NP_004272.2; Public GI: 14043024 is referred to herein as SEQ ID NO: 1. The NCBI reference nucleic acid sequence for BAG3 can be found at Genbank under accession number NM_004281.3 GI: 62530382. The nucleic acid sequence of Genbank accession number NM_004281.3 GI: 62530382 is referred as SEQ ID NO: 2. Other BAG3 amino acid sequences include, for example, without limitation, 095817.3 GI: 12643665 (SEQ ID NO: 3); EAW49383.1 GI: 119569768 (SEQ ID NO: 4); EAW49382.1 GI: 119569767(SEQ ID NO: 5); and CAE55998.1 GI: 38502170 (SEQ ID NO: 6). The BAG3 polypeptide of the invention can be a can be a variant of a polypeptide described herein, provided it retains functionality.

As used herein, “biological samples” include solid and body fluid samples. The biological samples used in the present invention can include cells, protein or membrane extracts of cells, blood or biological fluids such as ascites fluid or brain fluid (e.g., cerebrospinal fluid). Examples of solid biological samples include, but are not limited to, samples taken from tissues of the central nervous system, bone, breast, kidney, cervix, endometrium, head/neck, gallbladder, parotid gland, prostate, pituitary gland, muscle, esophagus, stomach, small intestine, colon, liver, spleen, pancreas, thyroid, heart, lung, bladder, adipose, lymph node, uterus, ovary, adrenal gland, testes, tonsils, thymus and skin, or samples taken from tumors. Examples of “body fluid samples” include, but are not limited to blood, serum, semen, prostate fluid, seminal fluid, urine, feces, saliva, sputum, mucus, bone marrow, lymph, and tears.

As used herein, “cardiac disease” refers to any type of heart disease including heart failure, heart muscle disease, cardiomyopathy, hypertrophic cardiomyopathy, dilated cardiomyopathy, atherosclerosis, coronary artery disease, non-ischemic heart disease, ischemic heart disease, myocarditis, viral infection, wounds, hypertensive heart disease, valvular disease, congenital heart disease, myocardial infarction, congestive heart failure, arrhythmias, diseases resulting in remodeling of the heart, etc. Diseases of the heart can be due to any reason, such as for example, damage to cardiac tissue such as a loss of contractility (e.g., as might be demonstrated by a decreased ejection fraction). Cardiac damage or disorder characterized by insufficient cardiac function includes any impairment or absence of a normal cardiac function or presence of an abnormal cardiac function. Abnormal cardiac function can be the result of disease, injury, and/or aging. As used herein, “abnormal cardiac function” includes morphological and/or functional abnormality of a cardiomyocyte, a population of cardiomyocytes, or the heart itself. Non-limiting examples of morphological and functional abnormalities include physical deterioration and/or death of cardiomyocytes, abnormal growth patterns of cardiomyocytes, abnormalities in the physical connection between cardiomyocytes, under-or over-production of a substance or substances by cardiomyocytes, failure of cardiomyocytes to produce a substance or substances which they normally produce, and transmission of electrical impulses in abnormal patterns or at abnormal times. Abnormalities at a more gross level include dyskinesis, reduced ejection fraction, changes as observed by echocardiography (e.g., dilatation), changes in EKG, changes in exercise tolerance, reduced capillary perfusion, and changes as observed by angiography. Abnormal cardiac function is seen with many disorders including, for example, non-ischemic or ischemic heart disease, e.g., angina pectoris, myocardial infarction, chronic ischemic heart disease, hypertensive heart disease, pulmonary heart disease (cor pulmonale), valvular heart disease, e.g., rheumatic fever, mitral valve prolapse, calcification of mitral annulus, carcinoid heart disease, infective endocarditis, congenital heart disease, myocardial disease, e.g., myocarditis, dilated cardiomyopathy, hypertensive cardiomyopathy, cardiac disorders which result in congestive heart failure, and tumors of the heart, e.g., primary sarcomas and secondary tumors. Heart damage also includes wounds, such as for example, knife wound; biological (e.g. viral; autoimmune diseases) or chemical (e.g. chemotherapy, drugs); surgery; transplantation and the like.

As used herein, “amyloidosis” refers to an abnormal buildup, increase, or accumulation of amyloid protein, such as B-amyloid in one or more cells, tissues, and/or organs and interferes with their normal function.

As used herein the phrase “diagnosing” refers to classifying a disease or a symptom, determining a severity of the disease, monitoring disease progression, forecasting an outcome of a disease and/or prospects of recovery. The term “detecting” may also optionally encompass any of the above. Diagnosis of a disease according to the present invention can be effected by determining a level of a polynucleotide or a polypeptide of the present invention in a biological sample obtained from the subject, wherein the level determined can be correlated with predisposition to, or presence or absence of the disease. It should be noted that a “biological sample obtained from the subject” may also optionally comprise a sample that has not been physically removed from the subject.

As used herein the phrase “diagnostic” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

An “effective amount” as used herein, means an amount which provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes: a recombinant DNA which is part of a hybrid gene encoding additional polypeptide sequence, complementary DNA (cDNA), linear or circular oligomers or polymers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like.

The nucleic acid sequences may be “chimeric,” that is, composed of different regions. In the context of this invention “chimeric” compounds are oligonucleotides, which contain two or more chemical regions, for example, DNA region(s), RNA region(s), PNA region(s) etc. Each chemical region is made up of at least one monomer unit, i.e., a nucleotide. These sequences typically comprise at least one region wherein the sequence is modified in order to exhibit one or more desired properties.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to a delivery systems comprising two or more separate containers that each contain a sub-portion of the total kit components. The containers may be delivered to the intended recipient together or separately. For example, a first container may contain an enzyme for use in an assay, while a second container contains oligonucleotides. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a sub-portion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

The term “target nucleic acid” sequence refers to a nucleic acid (often derived from a biological sample), to which the oligonucleotide is designed to specifically hybridize. It is either the presence or absence of the target nucleic acid that is to be detected, or the amount of the target nucleic acid that is to be quantified. The target nucleic acid has a sequence that is complementary to the nucleic acid sequence of the corresponding oligonucleotide directed to the target. The term target nucleic acid may refer to the specific subsequence of a larger nucleic acid to which the oligonucleotide is directed or to the overall sequence (e.g., gene or mRNA). The difference in usage will be apparent from context.

In the context of the present invention, the following abbreviations for the commonly occurring nucleic acid bases are used, “A” refers to adenosine, “C” refers to cytosine, “G” refers to guanosine, “T” refers to thymidine, and “U” refers to uridine.

Unless otherwise specified, a “nucleotide sequence encoding” an amino acid sequence includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Parenteral” administration of an immunogenic composition includes, e.g., subcutaneous (s.c.), intravenous (i.v.), intramuscular (i.m.), or intrasternal injection, or infusion techniques.

The terms “patient” or “individual” or “subject” are used interchangeably herein, and refers to a mammalian subject to be treated, with human patients one example. In some cases, the methods of the invention find use in experimental animals, in veterinary application, and in the development of animal models for disease, including, but not limited to, rodents including mice, rats, and hamsters, and primates.

The term “percent sequence identity” or having “a sequence identity” refers to the degree of identity between any given query sequence and a subject sequence.

The terms “pharmaceutically acceptable” (or “pharmacologically acceptable”) refer to molecular entities and compositions that do not produce an adverse, allergic or other untoward reaction when administered to an animal or a human, as appropriate. The term “pharmaceutically acceptable carrier,” as used herein, includes any and all solvents, dispersion media, coatings, antibacterial, isotonic and absorption delaying agents, buffers, excipients, binders, lubricants, gels, surfactants and the like, that may be used as media for a pharmaceutically acceptable substance.

The term “polynucleotide” is a chain of nucleotides, also known as a “nucleic acid” or “nucleic acid sequence” and include, but are not limited to, all nucleic acid sequences which are obtained by any means available in the art, both naturally occurring and synthetic nucleic acids, complementary DNA (cDNA), linear or circular oligomers or polymers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like. The nucleic acid sequences may be “chimeric,” that is, composed of different regions. In the context of this invention “chimeric” compounds are oligonucleotides, which contain two or more chemical regions, for example, DNA region(s), RNA region(s), PNA region(s) etc. Each chemical region is made up of at least one monomer unit, i.e., a nucleotide. These sequences typically comprise at least one region wherein the sequence is modified in order to exhibit one or more desired properties.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “transfected” or “transformed” or “transduced” means to a process by which exogenous nucleic acid is transferred or introduced into the host cell. A “transfected” or “transformed” or “transduced” cell is one which has been transfected, transformed or transduced with exogenous nucleic acid. The transfected/transformed/transduced cell includes the primary subject cell and its progeny.

To “treat” a disease as the term is used herein, means to reduce the frequency or severity of at least one sign or symptom of a disease or disorder experienced by a subject. Treatment of a disease or disorders includes the eradication of a virus.

“Treatment” is an intervention performed with the intention of preventing the development or altering the pathology or symptoms of a disorder. Accordingly, “treatment” refers to both therapeutic treatment and prophylactic or preventative measures. “Treatment” may also be specified as palliative care. Those in need of treatment include those already with the disorder as well as those in which the disorder is to be prevented. Accordingly, “treating” or “treatment” of a state, disorder or condition includes: (1) eradicating the virus; (2) preventing or delaying the appearance of clinical symptoms of the state, disorder or condition developing in a human or other mammal that may be afflicted with or predisposed to the state, disorder or condition but does not yet experience or display clinical or subclinical symptoms of the state, disorder or condition; (3) inhibiting the state, disorder or condition, i.e., arresting, reducing or delaying the development of the disease or a relapse thereof (in case of maintenance treatment) or at least one clinical or subclinical symptom thereof; or (4) relieving the disease, i.e., causing regression of the state, disorder or condition or at least one of its clinical or subclinical symptoms. The benefit to an individual to be treated is either statistically significant or at least perceptible to the patient or to the physician.

The term “variant,” when used in the context of a polynucleotide sequence, may encompass a polynucleotide sequence related to a wild type gene. This definition may also include, for example, “allelic,” “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. Of particular utility in the invention are variants of wild type gene products. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

A “vector” is a composition of matter which comprises an isolated nucleic acid and which can be used to deliver the isolated nucleic acid to the interior of a cell. Examples of vectors include but are not limited to, linear polynucleotides, polynucleotides associated with ionic or amphiphilic compounds, plasmids, and viruses. Thus, the term “vector” includes an autonomously replicating plasmid or a virus. The term is also construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Where any a nucleic acid sequence or an amino acid sequence is specifically referred to by a Swiss Prot. or GENBANK Accession number, the sequence is incorporated herein by reference. Information associated with the accession number, such as identification of signal peptide, extracellular domain, transmembrane domain, promoter sequence and translation start, is also incorporated herein in its entirety by reference.

### EXAMPLES

### Example 1: Amyloid is Increased in Hearts of BAG 3 +/− Mice Experiencing Heart failure with Preserved Ejection Fraction (HFpEF)

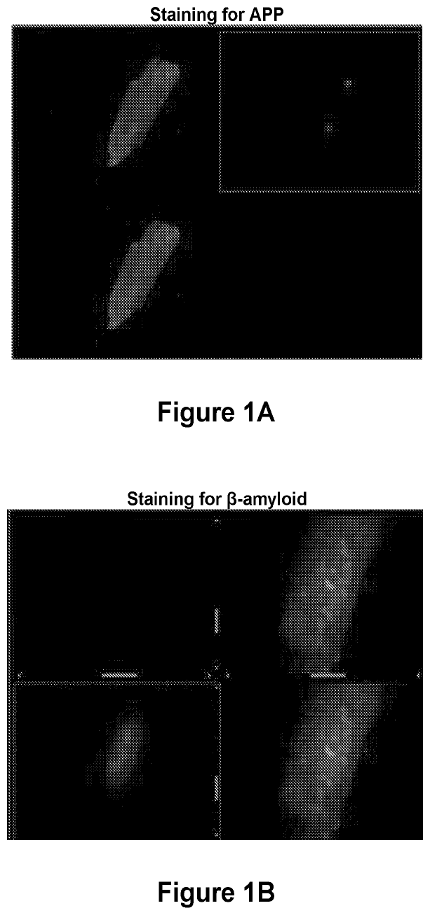

As depicted in FIG. 1, murine heart cells from BAG3 haploinsufficient (BAG3+/−) mice with were stained to visualize either APP or β-amyloid. The results indicate that β-amyloid in increased in cardiac cells of BAG3+/− mice.

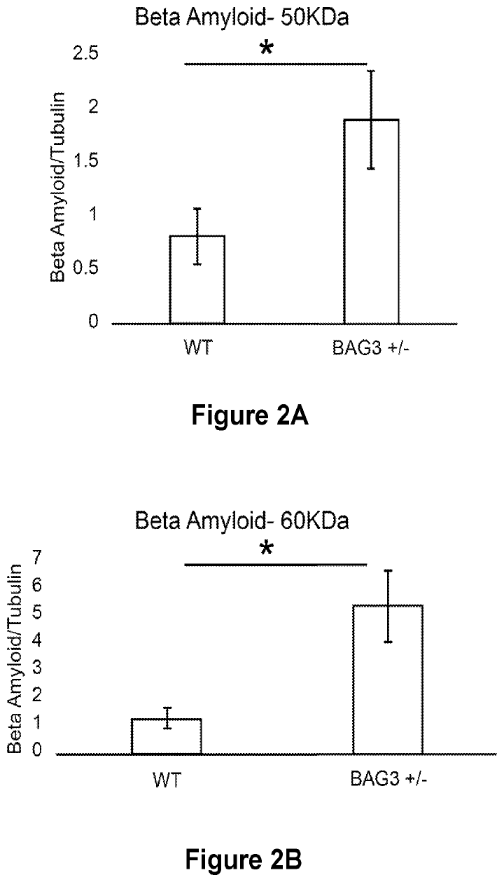

In FIGS. 2A and 2B, the amounts of β-amyloid normalized to amounts of tubulin in BAG3+/− mice compared to wild-type mice are provided.

The results indicated that-amyloid accumulated in cardiac cells of HFpEF mice to levels that greatly exceed those observed in wild-type mice.

1. A method of treating a patient suffering from, or at risk of developing, amyloidosis comprising:
administering to the patient a therapeutically effective amount of an agent wherein the agent modulates expression or amount of BCL2-associated athanogene 3 (BAG3) encoding nucleic acid, BAG3 protein or BAG3 peptide thereby treating amyloidosis.

2. The method of claim 1, wherein the BAG3 encoding nucleic acid comprises an expression vector expressing a BAG3 protein or active fragment thereof.

3. The method of claim 2, wherein the expression vector further comprises a promoter, the promoter comprising an inducible promoter, a constitutive promoter, bicistronic promoter or tissue specific promoter.

4. The method of claim 2 or 3, wherein the expression vector comprises a viral vector, cardiotropic vector, plasmid, or a yeast vector.

5. The method of any of claims 1-4, wherein a cardiotropic vector comprises an adenovirus vector, an adeno-associated virus vector (AAV), a coxsackie virus vector, cytomegalovirus vector, Epstein-Barr virus vector, parvovirus vector, or hepatitis virus vectors.

6. The method of claim 5, wherein the AAV vector comprises a capsid protein having 90% or more sequence identity to any of AAV1, AAV2, AAV3, AAV4, AAV5, AAV6, AAV7m AAV8, AAV9, AAV10, AAV11 or AAV12.

7. The method of claim 2 or 3, wherein the expression vector is a pseudotyped viral vector.

8. The method of any of claims 1-7, wherein the patient has or is at risk of having heart failure with preserved ejection fraction (HFpEF).

9. The method of any of claims 1-8, wherein the patient has or is at risk of having cardiac amyloid deposits or increased cardiac amyloid compared to age, gender and/or race matched control.

10. The method of any of claims 1-9, wherein the patient expresses lower than normal levels of BAB3 in cardiac tissue.

11. The method of any of claims 1-10, wherein the amyloidosis comprises cardiac amyloidosis or Alzheimer's disease.

12. The method of claim 11, wherein the patient does not have a mutation or defect in the transthyreitn (ATTAR) gene.

13. The method of any of claims 2-12, wherein the expression vector further comprises a promoter, the promoter optionally comprising an inducible promoter, a constitutive promoter, bicistronic promoter or tissue specific promoter.

14. The method of claim 13, wherein the inducible promoter confers expression in cardiac tissue or CNS.

15. The method of any of claims 2-14, wherein the expression vector further comprises an AAV inverted terminal repeat (ITR).

16. The method of any of claims 2-14, wherein the expression vector further comprises a polyadenylation sequence and/or stop codon.
